(19) United States
(12) Patent Application Publication
(10) Pub. No.: US 2004/0067241 A1
(43) Pub. Date: Apr. 8, 2004

(54) Comtrol a/k/a Virex antiviral composition

(76) Inventor: Gizelle T. Letourneau, Butte, MT (US)

Correspondence Address: Gizelle T. Letourneau, 1136 Caledonia Street, Butte, MT, 59701, US

(21) Appl. No.: 10/265,450

(22) Filed: Oct. 7, 2002

(51) Int. Cl. A61K 39/12; A61K 39/145; A61K 45/00; A61K 47/00

(52) U.S. Cl. 424/206.1; 424/204.1; 424/278.1

(57) ABSTRACT

An over-the-counter cold and flu medicine or alternative pharmaceutical formulation with other therapeutic actions. It is simply defined as a compound consisting of a phytopolymer (or a biopolymer or synthetic polymer), one or more microprotein solvents, a steroid, and a sugar—all of which may be of natural plant matter or their derivatives and/or chemical components.

### BACKGROUND OF THE INVENTION

The present invention relates to a new compound, derivatives of the formula and pharmaceutical formulations thereof, their use in medicine, and processes for their preparation. It concerns its use mainly as a cold and flu medicine, but is not limited as such. It is non-toxic as is verified herein.

Other similar inventions are only vehicles or carriers for therapeutic agents whereas Comtrol is a specific compound for use as a therapeutic agent. It is also not specific to certain cells like other inventions, but is a general biological treatment for use in or outside of any infected or diseased cell and as prophylaxis to the rest of the body. It is therefore not intended to target any specific cell.

Other inventions do not contain the combination of the three main groups of substances required to achieve Comtrol's specific actions as, for example, an antiviral.

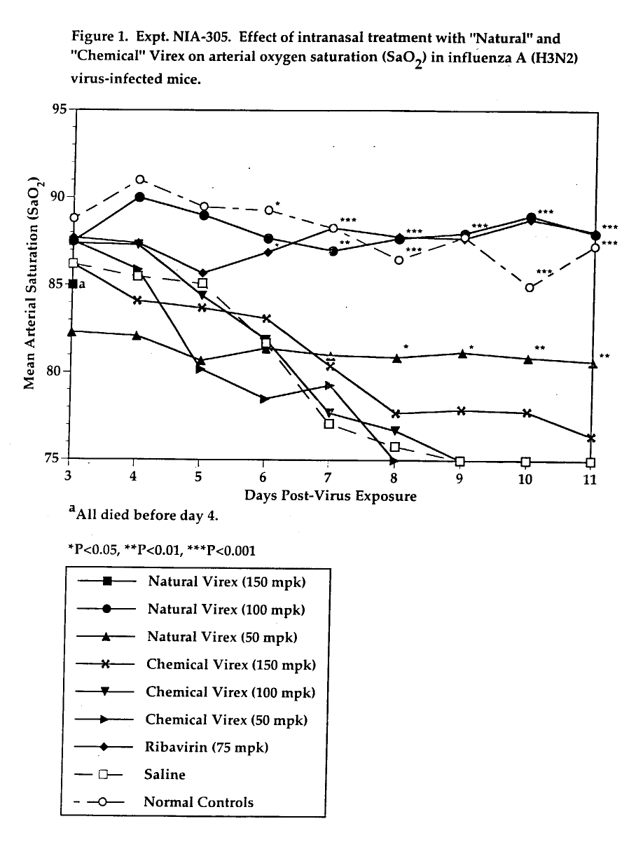

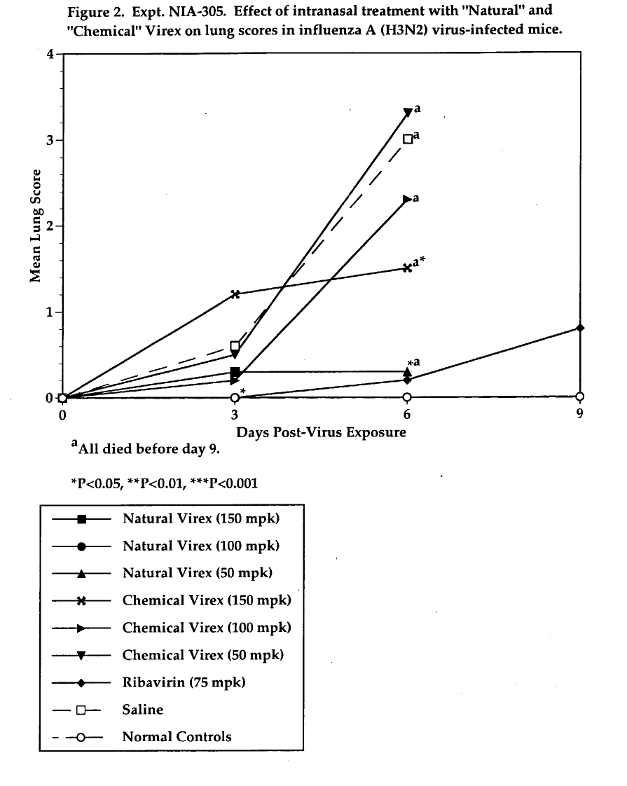

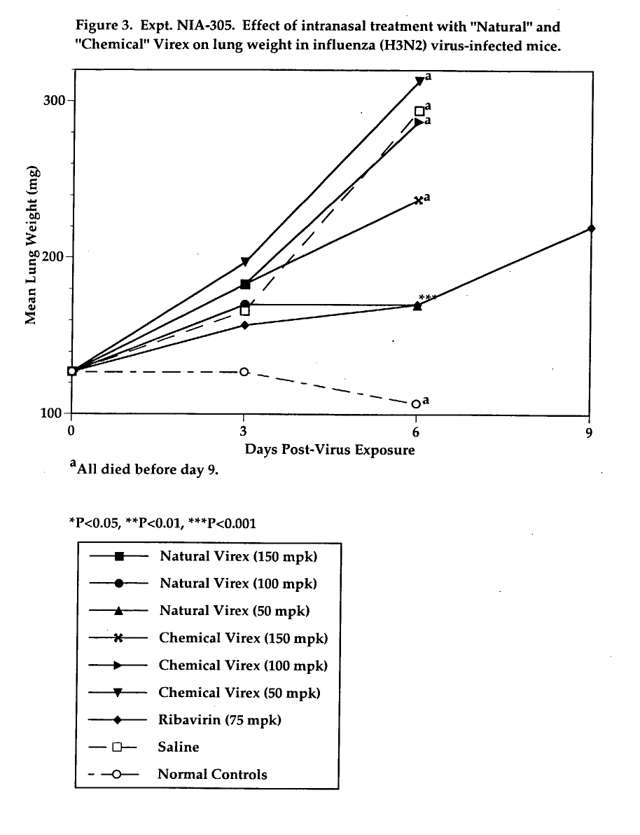

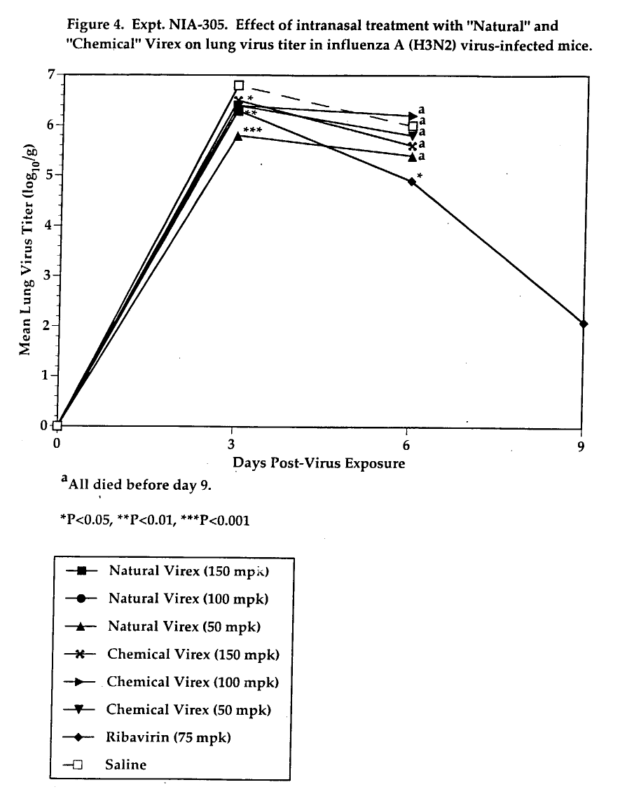

Comtrol is proven to have antiviral action against 100% lethal infection of Influenza H3N2 (Influenza A/Shangdong/09/93) in mice by the Institute for Antiviral Research, Logan, Utah—Table 1 and FIG. 14. It has also proven effective in preliminary studies by Eden Organic Complex Products against cold and flu. Glycyrrhiza glabra root, a main component of over-the-counter Comtrol, is known to be successfully used in treating encephalitis, which affects the brain. Encephalitis has become a major problem with the spread of the West Nile Virus in the United States. Glycyrrhizin and glycyrrhizic acid which are derivatives of glycyrrhiza root are reported to have antiviral activity against encephalitis (study cited above). It also has immunostimulating activity as well as having anti-tumor/anti-cancer, anti-allergic, anti-inflammatory and anti-ulcer activity (studies cited). Comtrol has very minimal side-effects which are a slight rise in blood pressure, pulse rate and decrease in potassium levels. It has shown no toxicity in animal studies as shown in Table F-2, Table G-2 and Table 1. (Comtrol may prove to have anticancer capacity as has been shown in preliminary studies by Eden Organic Complex Products and will be documented in a continuation of this patent. Comtrol may also be beneficial in the healing of serious burns that will be determined in future testing and described in a continuation of this patent.)

The effect of Comtrol a/k/a Virex was tested in vivo using mice infected with Influenza A/Shangdong/09/93 (H3N2). The control drug used was Ribavirin and normal controls were used as well. The results of the study are summarized in Table 1 and FIG. 14 attached herein. The mice for the studies were provided by the Centers for Disease Control. The infection induced in the mice was 100% lethal. At 50 mg/kg/day dose of Comtrol a/k/a Virex (in its synthetic alternate form as a pharmaceutical), there was a 50% increase in survivors (5 out of 10) of infected mice. At 100 mg/kg/day dose, 30% of the mice survived, however there had been a impediment due to an additive (for solubility) that caused the intransal administration of the compound to be problematic. (An additional study will be run excluding the additive with the results provided in a continuation in part of this patent.) The control drug exhibited 90% survival of the mice. All infected and untreated mice died. “These data confirm our previous findings that intranasally administered Virex will exert a significant inhibitory effect on experimentally induced influenza virus infections in mice. The results found in the present study are similar to those reported earlier using intranasally administration of this compound (Expt. NIA-281). “Robert W. Sidwell, Institute for Antiviral Research, Utah State University. National Institutes of Allergy and Infectious Disease research grant Jun. 6, 2002.

### SUMMARY OF THE INVENTION

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as the principles and conceptual aspects of the invention. It will be appreciated by those skilled in the art that derivatives of the ingredients of these compound or organic and/or biological chemicals may be formulated and modified to provide pharmaceutically acceptable alternative compounds producing the same essential compound as Comtrol.

### DETAILED DESCRIPTION OF THE INVENTION

Of particular interest as such derivatives or chemicals are a compound consisting of one or more of the substances from each of the following groups which must consist of at least groups one through three and preferably four which are 1) the polymer group such as mannose/mannitol, alanine or alpha-aminopropionic acid/2-aminopropanoic acid/3-aminopropanoic acid, albumin, arabinose, aldobionic acid, lactose, lactamic acid, lactamine, lecithin or phosphotidal choline-3, lectin, hexose, aldohexose, maltose, mucilage, succinic acid/succinate, gelatin, neoglycoproteins; 2) the microprotein solvent group such as adenine, coumarin, sapogenin, glucuronic acid, g lucuronate, retinol, retinoic acid, guanine, guaiacol, quinine/quinone, cinnamic acid, hydroxycinnamic acid, tyrosine; 3) the steroids group such as epinephrine, aldosterone, 17-hydroxycorticosterone, desoxycorticosterone, corticosterone, corticotropin, glycyrrhetinic acid, stigmasterol, B-sitosterol, solasodine, tomatodine, 17-hydrocorticosterone, diosgenin, “5, 22-cholestadien-24β-ethyl-3β-OL”; 4) the sugars group such as galactose/galactic acid, glucose/glucoronic acid, adenine or 6-aminopurine, aldose, ribose, deoxyribose, glycyrrhizic acid, glycyrrhinic acid, aldopentose; and 5) other biologicals or organics such as prostaglandin E2 and/or plant prostaglandins or lysozymes and/or plant lysozymes. Chemicals which could be added to increase activity would be sulfate, phosphate, bitartrate and nitrogen. Fortifiers and nutrient additives would be potassiums, antioxidents, carotene, cream of tartar, Vitamins A, K, D, E and/or C.

The initial compound is a syrup containing the water extract or powder of the root of Glycyrrhiza Glabra (a natural steroid and microprotein solvent which also comprises sugar(s), hereinafter referred to as “glycyrrhiza”) 20-25% and that of Althea root 15-20% and the powdered gum of the Acacia tree (a phytopolymer) in water 15-20% and corn syrup 40% or a pharmaceutically acceptable carrier or diluent with mint extract 0.01-0.04%, and salt or other suitable preservative such as potassium sorbate. (It may contain the minimal level of one of the FDA designated active ingredients for cold and flu medicines for their indications. However, it may also be made without such known active ingredients as mainly a natural medicine for the strengthening of mucous membranes and the immune system. It has been proven as an antiviral and approval for an antiviral indication will be sought from the FDA.)

A synthetic alternate and preferred pharmaceutical compound comprised of the first four types of substances is −45% coumarin, 20% mannose, 9% glucuronic acid, 19% galactose, 7% “5, 22-cholestadien-24β-ethyl-3β-OL”, all of which are active ingredients according to the inventor, and 0.01 to 0.05% potassium sorbate, or other suitable preservative (i.e. sodiums or citrates), suspended in corn syrup and water. Concentration is 55 mg/ml of active ingredient. This compound has been proven by the Institute for Antiviral Research to behave as an equal to the initial compound and is therefore a synthetic reproduction of the initial compound described previously (containing glycyrriza glabra root, etc.). It performs, in fact, superior to the former compound in prevention of death to mice which received terminal doses of flu virus.

The over-the-counter version can be made in a gel form for topical use, or as a saline based nasal spray or eye drop, an inhalant, chewable tablet, a liquid gelcap, injection or an intravenous medicament.

A pharmaceutically acceptable alternative suspension (such as in the form of a gel or gelcap) may provide additional stability or synthesis. Pharmaceutically acceptable salts of the compounds of this formula include those derived from inorganic and organic acids and bases. It may be also be preferable to provide a compound excluding the sugars and certain amino acids to treat patients taking psychotropic medications due to the nature of the sugars and acids that enable the compound to cross the blood-brain barrier. This would prevent any interaction with other medications taken by such a patient as well as interactions with their condition when they are being treated for viral infection or disease. However it would not be preferable for normal treatment of patients with no psychological dysfunction since flu infection can progress to encephalitis of the brain.

Dosage is preferably 12 to 26 mg/kg of body weight per dose but is not limited to such dosage levels. It will be appreciated that reference herein to treatment extends to prophylaxis as well as the treatment of established infections or symptoms. However, the medicine is normally used to treat the symptoms once they have appeared. The amount of compound of the invention required for use in treatment will vary with the route of administration requiring various forms of the compound, i.e. inhalant, etc.; the nature of the condition being treated; and the age and condition of the patient. Ultimately dosage will be at the discretion of the attending physician or veterinarian up to the maximum non-toxic dose tested by the Antiviral Institute of Utah State University of 50 to 100 mg/kg body weight for both the over-the-counter and pharmaceutical formulas specifically provided herein. Treatment is preferably commenced at the first sign of infection.

Microprotein solvents listed herein are described by Blackiston's New Gould Medical Dictionary as follows:

retinol: A liquid hydrocarbon obtained in the destructive distillation of resin. It is used as a solvent and was formerly employed in gonorrhea;

guiaiacol: A liquid usually obtained from wood creosote which is caustic. (or a solvent)

Reference to coumarin or Coumadin as a microprotein solvent is elucidated by the fact that it is known to prevent thrombosis caused by enzymes and is possibly an enzyme inhibitor. Glucuronic acid and like or related substances break down dead tissue cells in the human body and are therefor also solvents.

The mode of action of these substances is the breakdown of viral protein, a direct antiviral effect, reflective in the study performed using Comtrol at the Institute for Antiviral Research. Other actions are the possible breakdown of repressor protein involving Lex A and Rec A in DNA, activating repair mechanisms. The phytopolymers combined with a solvent, steroid and a sugar (or polysaccharide) are most likely penetrating and strengthening membrane tissues (such as cell walls) and cellular as well as nuclear signaling. “This polysaccharide class of biopolymers is important for the structural and functional roles in cell walls of bacteria and plants and in the cell membranes of animals . . . and in addition, some polysaccharides (glycyrrhiza is a polysaccharide) are important forms of fuel storage. The hexoses are by far the most important naturally occurring sugars, although aldopentoses are important components of nucleic cells.” Chemical Encyclopedia Vol. 3 Some of these substances are also known to be enzyme inhibitors and may account for some of its antiviral and anticancer actions.

Some of the steroids listed herein are described by An Introduction to Genetic Analysis as follows:

“Small hormones and, in particular, steroid hormones enter cells by virtue of their lipid-solubility and bind to receptors in the nucleus. . . . The estrogen molecule activates transcription by binding to the DNA at an enhancer site, together with a protein receptor molecule that first recognizes the estrogen molecule in the cytoplasm and transports it to the nucleus.”

Thus, it is likely that the steroid component of Comtrol acts as the vehicle taking the microprotein solvents to the nucleus to do their work, i.e. dissolving viral or bacterial DNA which is smaller than human DNA and therefore more vulnerable. (DNA research is planned and will be contained in a continuation of this patent.)

Pharmaceutical formulations include those suitable for oral, nasal, topical, parenteral (including intramuscular, subcutaneous and intravenous injection), rectal, vaginal administration or as an inhalant or insufflation. The formulations may, where appropriate, be conveniently presented in individual dosage units and may be prepared by any of the methods well known in the art of pharmacy. All methods included the step of bringing into association the active compound with liquid carriers or finely divided solid carriers or both and then, if necessary, shaping the product as a pill, caplet or other medium. The compound may be presented as a bolus, electuary or paste. Tablets and capsules may contain conventional excipients such as binding agents, fillers, lubricants, disintegrants, wetting or gelling agents. Tablets may be coated according to methods well known in the art. A chewable tablet may be formed which may contain coloring or flavoring agents. Liquid preparations may be in the form of, for example, aqueous, gelatinous or synthetically gelatinous suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles, coloring agents, flavoring agents or preservatives.

The compounds according to the invention may also be formulated for parenteral administration (e.g. by injection, for example, bolus injection or continuous infusion) and may be presented in unit dose form in ampoules, prefilled syringes, small volume infusion or in multi-dose containers with an added preservative. The compositions may take such forms as suspensions, solutions, or emulsions in gelatinized, aqueous or oily vehicles, and may contain formulatory agents such as suspending, stabilizing and/or dispersing agents. Alternatively, the active ingredients may be in powder form, obtained by aseptic isolation of sterile solid or nitrogen laser concentration from solution, for constitution with a suitable vehicle, e.g. sterile, pyrogen-free water, before use. However, generally this compound is not to be frozen as it will lose beneficial characteristics.

For topical administration to the epidermis the compounds according to the invention may be formulated as ointments, hydrogels, gel-packs, creams or lotions, or as a transdermal patch or gel filled membrane bandages. Ointments, hydrogels and creams may, for example, be formulated in an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations for administration in the mouth include lozenges comprising active ingredients in a flavored base, usually sucrose and acacia or tragacanth; pastilles comprising the active ingredients in a base such as gelatin and glycerin or sucrose and acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the carrier.

Formulations for vaginal administration may be presented as pessaries, gels, pastes, foams or sprays containing in addition to the active ingredients such carriers as are known in the art to be appropriate.

Thus, in general, the compounds may be administered in the form of a solution or a suspension or as a dry pill or powder.

Solutions and suspensions will generally be aqueous for example prepared from water alone (for example sterile or pyrogen-free water) or water and a physiologically acceptable solute (for example ethanol, glycerin, propylene glycol, polyethylene glycols).

Such solutions or suspensions may additionally contain other excipients, for example, preservatives, solubilizing agents/surfactants such as polysorbates, buffering agents, isotonicity-adjusting agents, absorption enhancers, suspending agents and viscosity enhancers.

Solutions or suspensions are applied directly to the nasal cavity or other mucous membrane surface by conventional means, for example, with a dropper, needleless syringe, pipette or spray/aerosol. The formulations may be provided in single or multidose form. In the latter case a means of metering is desirably provided which is achieved by the patient or care provider administering an appropriate, predetermined volume of the solution or suspension. In the case of a spray this may be achieved for example, by means of a metering atomizing spray pump.

For intranasal administration according to the method of the invention the active ingredients may be administered by any of the methods and formulations employed in the art for intranasal administration. Intranasal administration may also be achieved by means of an aerosol formulation in which the compound is provided in a pressurized pack with a suitable propellant such as a chlorofluorocarbon for example dichlorodifluoromethane, trichlorofluoromethane or dichlorotetrafluoroethane, carbon dioxide or other suitable gas. The aerosol may conveniently also contain a surfactant such as lecithin. The dose of drug may be controlled by provision of a metered valve. The compound for use as such will generally have a small particle size for example of the order of 5 microns or less. Such a particle size may be obtained by means known in the art, for example by micronization.

When desired, formulations adapted to provide sustained release of the active ingredients may be employed.

Alternatively the compounds may be added to a nebulizer for inhalation in proper dosage amounts.

A saline solution containing the highly filtered liquid compound is suitable for administration to the eye with any pharmaceutically acceptable additive.

TABLE 1
Expt. NIA-305. Effect of Intranasal Treatment with High Dosages of
“Chemical” and “Natural” Virex on an Influenza A (H3N2)
Virus Infection in Mice.

| | |
|---|---|
| Animals: Female 18-21 g BALB/c mice | Treatment schedule: bid × 5 beg.4 h pre-virus |
| Virus: Influenza A/Shangdong/09/93 (H3N2) | exposure |
| Drug diluent: 0.4% CMC (“Chemical” Virex), | Treatment route: i.n. |
| saline (“Natural” Virex) | Expt. duration: 21 days |

| | | Tox Controls | | Infected, Treated Mice | | |
|---|---|---|---|---|---|---|
| | Dosage | Surv/ | Mean Host Weight | Surv/ | Mean Day to | Mean Day 11 |
| Compound | (mg/kg/day) | Total | Change^{a}(g) | Total | Death^{b} ± SD | SaO₂(% ± SD) |
| “Chemical” Virex | 150 | 0/3 | ?^{c} | 0/9 | 2.8 ± 0.8 | ?^{c} |
| | 100 | 0/3 | ?^{c} | 3/10* | 1.7 ± 1.5 | 88.0 ± 1.8*** |
| | 50 | 3/3 | −0.8 | 5/10** | 2.8 ± 1.5 | 80.7 ± 6.1** |
| “Natural” Virex | 150 | 3/3 | −0.9 | 1/9 | 7.3 ± 2.3 | 76.4 ± 4.3 |
| | 100 | 3/3 | −0.2 | 0/10 | 7.0 ± 1.4 | 75.0 ± 0.0 |
| | 50 | 3/3 | 0.0 | 0/10 | 6.4 ± 1.0 | 75.0 ± 0.0 |
| Ribavirin | 75 | 3/3 | −1.7 | 9/10*** | 2.0 ± 0.0 | 88.1 ± 1.3*** |
| Saline | — | — | — | 0/10 | 6.9 ± 1.1 | 75.0 ± 0.0 |
| Normal Controls | — | 3/3 | 0.6 | — | — | — |

^{a}Difference between initial weight and weight 18 h after final treatment.
^{b}Mean day to death of mice dying prior to day 21.
^{c}Unknown due to toxicity-related early death of mice.
*P < 0.05; **P < 0.01; ***P < 0.001 compared to saline-treated controls.

What is claimed is as follows but is not limited to:

1. An antiviral which prevents death as well as the symptoms in some viral infections, especially influenza. This is achieved by destroying or making latent the virus due to the action of the solvent. It also increases the immune response.

2. An antibacterial (data to be provided in a continuation of this patent).

3. An anti-cancer medicine (data to be provided in a continuation of this patent).
